(19) United States
(12) Patent Application Publication
(10) Pub. No.: US 2003/0072674 A1
(43) Pub. Date: Apr. 17, 2003

(54) Microwaveable container for sterilizing objects

(76) Inventors: Richard Melker, Gainesville, FL (US); Nikolaus Gravenstein, Gainesville, FL (US)

Correspondence Address: VAN DYKE & ASSOCIATES, P.A., 7200 LAKE ELLENOR DRIVE, SUITE 252, ORLANDO, FL, 32809, US

(21) Appl. No.: 09/978,898

(22) Filed: Oct. 16, 2001

(51) Int. Cl. A61L 2/00; B01J 19/08

(52) U.S. Cl. 422/21; 422/186; 422/307; 250/455.11

(57) ABSTRACT

A method and device for sterilizing objects in a microwave oven is disclosed. The invention comprises an inexpensive container for sterilizing medical devices and other objects used in a home that have the potential to transmit infectious diseases. An indicator, preferably responsive to heat, to indicate when an object has been adequately sterilized, is placed on, or incorporated into, the container to alert a user that an object has been properly sterilized.

### FIELD OF THE INVENTION

The present invention generally relates to method and devices for sterilizing objects, and more particularly to sterilizing household objects and medical equipment in a microwave oven.

### BACKGROUND

Bacteria, fungi, viruses, prions and other microbes have successfully colonized nearly every environment on Earth, including human dwelling places and the human body. For the most part, a harmless symbiotic relation exists between microbes and humans. However, occasionally organisms that are pathogenic, such as species of Salmonella, E. coli, Shigella, Campylorbacter. Clostridium and Staphylococcus are introduced into an individual. Every year, more than 7 million Americans fall victim to microbial infections which overwhelm the body's natural defenses and manifest as flu-like symptoms or, in extreme cases, even death. For immuno-compromised individuals, exposure to even normally harmless microbes may prove lethal. Many of these infections originate in the household kitchen.

Despite having one of the safest food supplies in the world, over 75 million people become ill and nearly 5,000 people die each year in the United States from exposure to food borne bacteria. (Center for Disease Control, 1999). Food contamination may result from unsanitary practices in the kitchen. A major cause of this problem can be traced to improper cleaning techniques or re-use of contaminated objects, such as a sponge or dishrag to clean an area. Over time, these objects can become a significant source of food borne infection by spreading harmful bacteria over large areas. Because they remain moist for prolonged periods of time and many have foodstuffs on or in them, sponges and other household items used to clean an area can support the growth of a wide assortment of organisms, some of them potentially lethal. For example, in one study conducted by the University of Arizona, sponges and dishrags used repeatedly to clean counters, spills or the like contained dangerous levels of the bacteria Salmonella or Stapylococci—two of the leading causes of food-borne illness. In another study, two-thirds of sponge samples tested positive for coliform bacteria and one-third tested positive for species of E. coli bacteria. (Josephson et al. 1997 Journal of Applied Microbiology 83:737-758). Another study reported that the use of detergents in hot water to clean a kitchen environment did not reduce the level of microbial contamination (Scott et al. 1984 Journal of Hygiene 92:193-203) Some infectious disease experts have recommended periodic washing of sponges in the dishwasher, but to date there are no studies documenting the efficacy of this strategy.

Another common source of microbial infection is re-use of objects placed within the body. For example, when a toothbrush is not properly cleaned and stored, food particles, toothpaste residue and other substances remain, allowing a variety of bacteria and viral agents to survive for substantial periods of time within the bristles. (Glass RT. 1998, J. Am Dent Assoc., 129(8):1076) Recent clinical studies have indicated that billions of harmful pathogens such as, for example, influenza viruses, herpes simplex I, Streptococcus, Staphylococcus, Candida, Gingivalis and other microorganisms that cause gum disease, colonize a toothbrush, providing an opportunity for these harmful agents to be re-introduced into the oral cavity with each brushing. For example S. mutans and other pathogenic microorganisms have been shown to be readily transferred when a toothbrush is used, increasing the risk of dental caries and infectious diseases. (Kozai K., et al. 1989 ASDC J Dent Child, 56(3): 201-4) Further, some bacteria may enter the bloodstream during certain oral hygiene measures, especially in patients with advanced chronic gingival disease. (Gillette W B. and R L J Van House, 1980 J. Am Dent Assoc 101(3):476-80) Methods to clean toothbrushes typically include chemical treatment that may leave harmful residue on the brush, or ozone treatment requiring expensive equipment. Often, because known methods of cleaning are time consuming and arduous, individuals use a toothbrush for an extended period of time and later discard it without ever having it sterilized.

An emerging cause of microbial infection is improper sterilization of home-care medical equipment. With the length of an average hospital stay decreasing and preference to receive care at home increasing, the home medical industry continues to experience substantial growth. In 1999, an estimated 20,000 providers delivered home care services to over eight million individuals at a cost of approximately $36 billion. (U.S. Department of Census, 1999; National Association for Home Care; 1999). Many home care patients have treatment regimes that require insertion of medical equipment within a body cavity, placing them at great risk of infection. For example, many people with para- or quadriplegia must self catheterize themselves several times each day with a Foley catheter. Chronically used devices such as tracheotomy tubes, gastrotomy tubes, peritoneal dialysis catheters, and numerous other medical devices become easily contaminated with bacteria and can be a chronic source of infection. For example, many microorganisms form a living layer of cells. Microorganisms generally form biofilms on biological surfaces, such as, for example, intestinal mucosa, and non-biological surfaces, such as, for example a catheter or a catheter bag. These “biofilms” not only may lead to urinary tract infections, but also are associated with encrustation and catheter blockage. (Godfrey H, and A. Evans, 2000 Br J Nurs, 9(11): 682-4, 686, 688-90). Urinary tract infection rates of 2.8 to 4.5 per 1,000 catheter days have been reported. (Luehm D, Fauerbach L. 1999 Caring, 18:30-4.) (Rosenheimer L., et al., 1998 Am J Infect Control, 26:359-63.) Further, rates of intravenous catheter-related bloodstream infections have been reported to range from 1.1 to 4.2 per 1,000 catheter days (Rosenheimer L., et al. 1998 Am J Infect Control, 26:359-63; White M C., et al., 1994 Am J Infect Control, 22:213-35) Added to this problem is the fact that many home-care patients are immuno-compromised. Opportunistic organisms that are usually relatively harmless can be dangerous, even lethal, to sick patients. For example, patients using gastrotomy or nasogastric tubes taking various medications for acid reflux disease may develop serious infections from using contaminated tubing since the acid in the stomach which is normally a significant barrier to bacterial infection, is neutralized. By providing patients with clean, sterile medical equipment, home care providers can contribute to improved quality of life of each patient while simultaneously reducing the cost of health care as secondary medical complications associated with infection are eliminated or reduced. Proper sterilization of re-usable devices can help the home care patient stay as healthy as possible. For example, sterilization of medical devices has been shown to reduce the incidence of urinary tract infection (UTI). In one study, patients using sterile catheterization techniques had a 28.6% UTI incidence while another group using a non-sterile catheterization technique had a UTI incidence of 42.4%. (Prieto-Fingerhut T, 1997 Rehab. Nurs., 22(6): 299-302) Traditional means to sterilize (or reduce the bacterial load of) home medical devices include using microwave ovens. Many patients with urinary tract pathology resulting in urinary retention microwave their catheters periodically because microwaves have been shown to effectively kill bacteria, fungi, spores and bacterial phages inhabiting them. (Mervine, J. and R. Temple, 1997 Rehab. Nurs. 22(6):318-320) However, simply exposing a medical device to microwave energy is often inadequate and may cause damage. Because different microwave ovens have wide variations in output, there is presently no means to document sufficient time in the oven and as a result equipment, such as, for example catheters, may be under or over treated. Inadequate treatment may leave live organisms in or on the devices. Excess treatment may lead to degradation of the material over time, particularly for those items constructed of materials having a low melting point. Thus, a need exists in the field for an efficient, cost-effective method and apparatus to adequately sterilize household objects and medical devices without causing damage to the material.

### SUMMARY OF THE INVENTION

The present invention provides an inexpensive microwaveable container for sterilizing medical devices and other objects used in a home that have the potential to transmit infectious diseases. An indicator, preferably responsive to heat, is placed on, or incorporated into, the container to indicate when an object has been adequately sterilized. According to one version of the subject invention, the container is constructed of a microwaveable material having a bi-concave shape that may be attached along one side by a hinge so as to open like a clam shell to reveal an interior chamber, or alternately may be constructed of two pieces which mate. A variety of household objects or medical devices that can become contaminated with microbes may be placed within the internal chamber for sterilization within a microwave oven. These and other features of the present invention are discussed in the following detailed description of the preferred embodiments of the present invention.

### BRIEF DESCRIPTION OF THE DRAWINGS

FIG. 1A is an illustration of a first embodiment of the present invention when closed.

FIG. 1B is an illustration of a first embodiment of the present invention when open.

FIG. 2A is an illustration of a second embodiment of the present invention when closed.

FIG. 2B is an illustration of a second embodiments of the present invention when open.

FIG. 3A is an illustration of a first embodiment of the present invention showing an indicator placed on the outside. Also shown is a change in color of the indicator that indicates that proper sterilization has been achieved.

FIG. 3B is an illustration of a first embodiment of the present invention showing a container having an indicator imbedded within the material used to construct the container. Also shown is a representative color change of the container to show that proper sterilization has been achieved.

FIG. 4A is an illustration of one embodiment of a removable stand for receiving or supporting an object to be sterilized.

FIG. 4B is an illustration of a first embodiment of the present invention in an open position to show placement of a stand within the interior chamber.

FIG. 5A is an illustration of an alternate embodiment of the present invention in a closed position showing a water chamber and pressure relief valve open to indicate sterilization has been achieved.

FIG. 5B is an illustration of an alternate embodiment of the present invention in an open position showing a water chamber and pressure relief valve incorporated as an indicator of sterilization.

FIG. 6A is an illustration of an alternate embodiment of the present invention position having only a water chamber, shown in a closed position, that functions as a pressure cooker.

FIG. 6B is an illustration of an alternate embodiment of the present invention in an open position having only a water chamber.

### DETAILED DESCRIPTION OF THE INVENTION

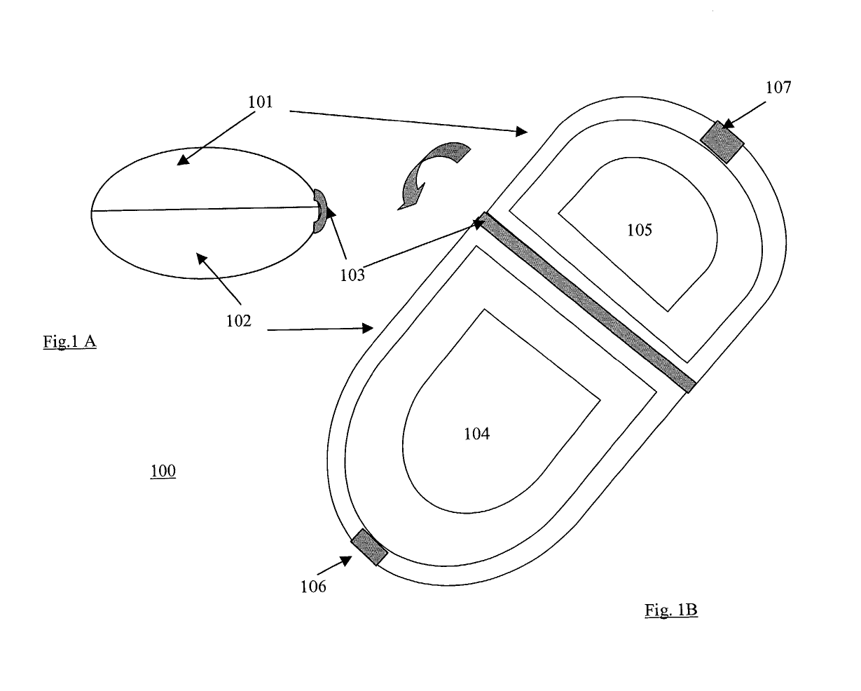

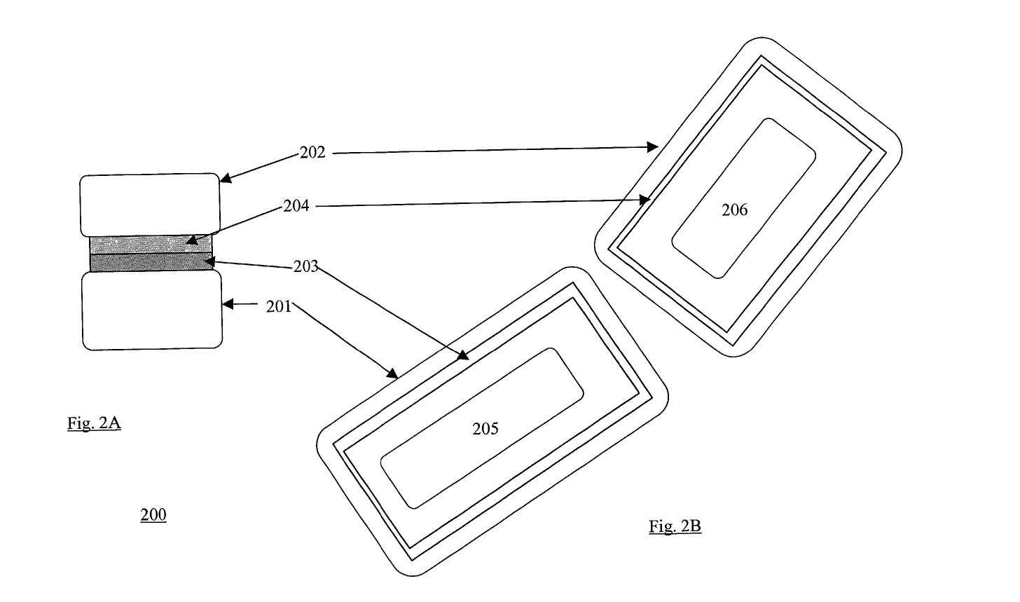

The present invention provides an inexpensive microwaveable container into which a contaminated object may be placed for sterilization. FIG. 1A illustrates a closed, first embodiment of a container for home sterilization of medical devices and household objects according to the present invention. The container generally represented at 100 comprises a top portion 101 and bottom portion 102 connected by a hinge 103. The material is made of a material that is at least partially transparent to microwaves. FIG. 1B illustrates a first embodiment the container of the present invention in an open position. A top portion 101 and bottom portion 102 connected by a hinge 103 is opened to reveal an internal bottom chamber 104 and an internal top chamber 105. In use an object to be sterilized is placed inside the bottom chamber 104. The container is closed and sealed by attaching a clasp 106 to a receiver 107. When closed, the container forms a gas tight seal. The container holding an object(s) to be sterilized is then placed in a microwave whereupon it is subjected to sufficient temperature for a sufficient period of time to be adequately sterilized. Bacteria are killed by exposure to the increase in temperature caused by absorption of microwaves. Because the organisms themselves absorb few microwaves, a suitable microwave absorbing materials can line one or both sides of the container to reach higher temperatures. Alternatively, an object placed inside can be rinsed and left wet. Microwave energy will cause the water to boil, which generally is sufficient to kill most organisms. FIGS. 2A and 2B show an alternate embodiment of the container of the present invention, generally indicated at 200, wherein the container is constructed of two pieces, a bottom removable portion 201 and a top removable portion 202. A bottom edge 203 mates with a top edge 204 to form a gas tight seal. Objects to be sterilized may be placed within a bottom interior chamber 205 or a top interior chamber 206 and then covered by the remaining portion prior to being placed within the microwave.

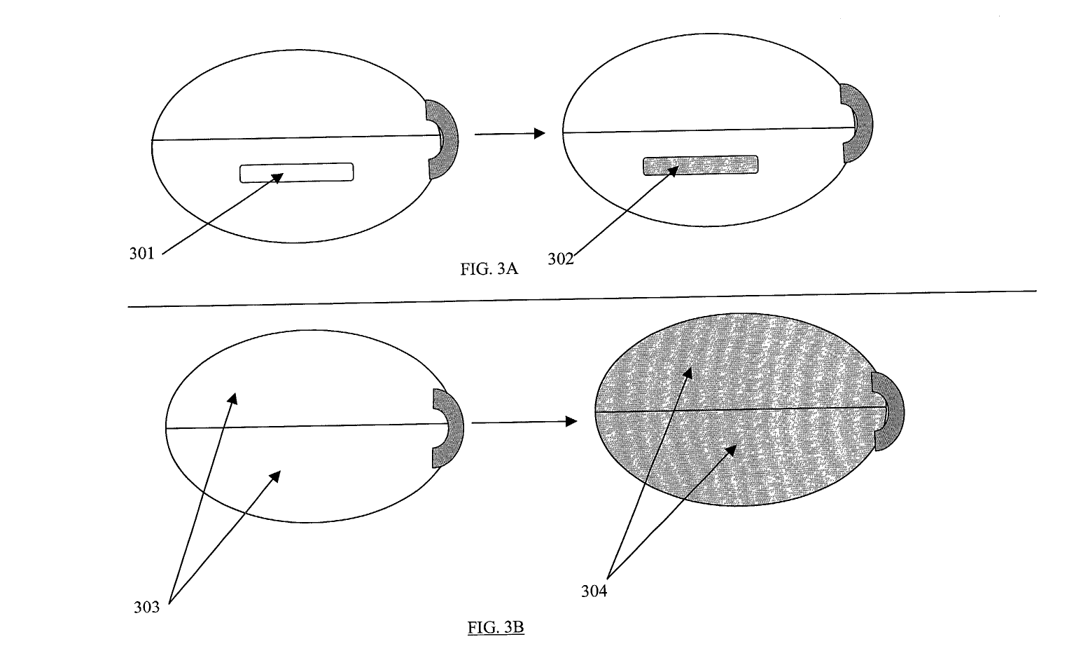

Preferably, all embodiments of the container utilize a indicator which either changes color, emits a sound, or both when the container has reached sufficient temperature for a sufficient period of time to achieve proper sterilization of an object placed therein. Use of an indicator provides significant advantage over other methods of sterilization because it allows a user achieve proper sterilization without damaging the object. This is particularly valuable when sterilizing objects with low melting points, such as plastics, which might become warped or burned with excess exposure to microwaves. FIG. 3A shows one embodiment of the present invention having a color change indicator strip placed on the outside of the container 100 (see FIG. 1). As shown, the colorless indicator strip 301 is activated to become a colored indicator strip 302 when objects placed within the chamber have been properly sterilized. FIG. 3B shows an alternative embodiment wherein the container is constructed with a material having a color indicator embedded within it. As shown, the colorless shell 303 becomes a colored shell 304 following activation of the indicator to signal that objects contained therein have been properly sterilized. It should be understood that various modifications to this concept are contemplated. For example, as an alternative to placing a color indicator on or in the container, one could design sponges, toothbrushes, medical devices or the like that have a color change indicator placed on or built into them that changes color after sufficient time in a microwave oven to kill contaminating organisms. Thus, the sponge itself, or an indicator on a toothbrush can change color to indicate when it has been properly sterilized. A cutting board with a color indicator that is microwave safe is another embodiment, since cutting boards have been shown to be a significant source of contamination and infection.

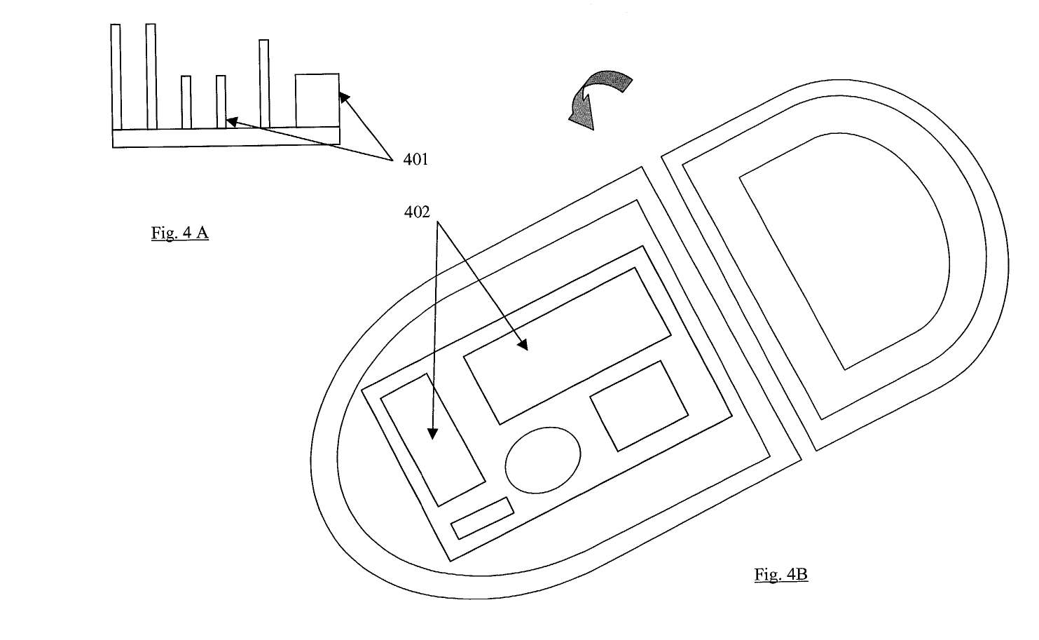

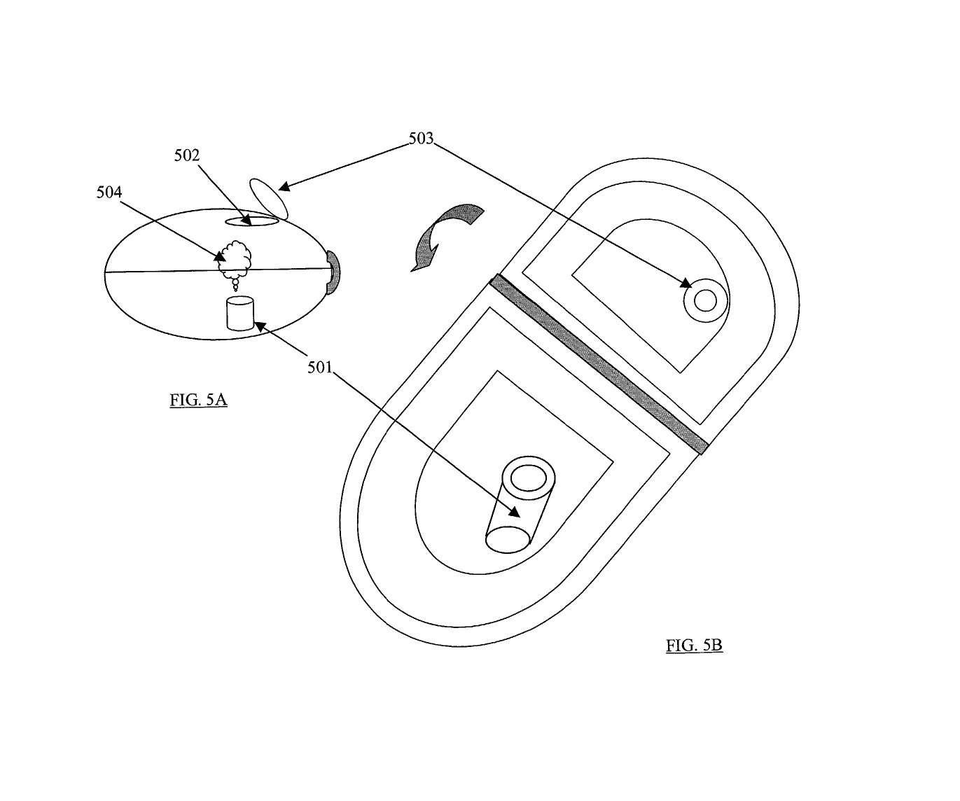

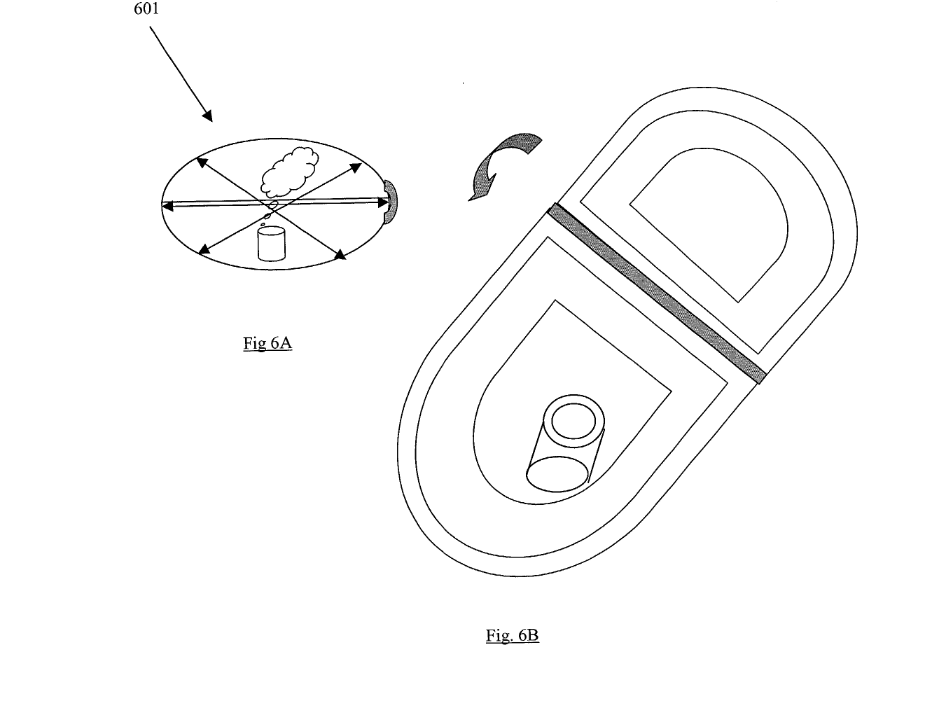

FIG. 4A shows one embodiment of a removable stand having supports 401 and wells 402 used to support or receive various objects placed within the container. Alternate designs may incorporate custom slots for specific medical devices, or other household objects such as sponges, toothbrushes, etc. FIG. 4B is an illustration of an open container showing placement of the removable stand within the interior chamber. FIG. 5A shows an embodiment of a closed container having a water compartment 501 and a pressure valve 502 capable of producing an acoustic signal when opened. FIG. 5B shows the same container in an open position. The water compartment 501 is placed within the lower chamber 104 (see FIG. 1) of the container and filled with water. In use an object is placed inside the lower chamber, the container is sealed and placed in a microwave. When sufficient pressure is produced inside the chamber from water boiling in response to the absorption of microwaves, the pressure valve 502 opens and sound produced from the escaping gas warns a user that the object(s) placed therein has been adequately sterilized. An alternative to this design is shown in FIGS. 6A and 6B, wherein a water compartment 501 is included, but the pressure valve is absent. Pressure builds up inside and presses against the walls of the container (generally indicated by 601) to act as a pressure cooker. When sufficient pressure is built up, a visual indicator alerts a user that sterilization has been achieved. In this embodiment, the internal environment of the container functions similar to an autoclave. The application of moist heat (saturated steam) under pressure damages cell structure, including the cytoplasmic membrane, rendering cells non-viable. Thus, the present invention provides an effective, economical means for killing a wide variety of microorganisms, thereby sterilizing the object placed inside the chamber. Other chemicals having bactericidal or sporicidal effects may also be placed into the container to aid with the sterilization procedure if desired.

Materials suitable for use in construction of the container of this invention include any microwaveable material which is at least partially transparent to microwave energy, and which exhibits no permanent deformation upon heating. Preferably, the container is made from re-usable inexpensive plastics, such as polyethylene, polyethylene terephthalate, polypropylene, epoxies and the like. Other materials suitable for construction include microwaveable metals, such as for example, aluminum; and microwaveable glass and ceramics, such as for example, oven glass pyroceram (glass-ceramic), porcelain and the like.

Any thermochromic material (i.e. capable of changing color in response to temperature) may be used as an indicator. Preferably, the indicator of the present invention is composed of a mixture of thermally changeable pigments which is either packaged in an adhesive strip or integrated with the material used to form the container of the present invention. U.S. Pat. No. 5,085,801 issued to Centre National de la Recherche Scientifique on Feb. 4, 1992, discloses an indicator for detecting whether the temperature of an article has exceeded a selected temperature threshold. The indicator comprises a chemically inert compound exhibiting irreversible thermochromism so that the compound undergoes a change from a blue color to a red color at the selected temperature. U.S. Pat. No. 4,339,951 issued to Allied Corporation discloses use of thermochromic polyacetylenes in temperature-indicator and indicia-display device applications. These polyacetylenes exhibit reversible color changes at transition temperatures in the range −180° to 220° C., wherein the thermochromic cycles can be repeated many times with no apparent degradation and little change in spectroscopic properties. WO 112,148A1 issued to Hindustan Lever Ltd. on Feb. 22, 2001 discloses cosmetic strips with liquid crystal temperature dependent color change. The adhesive strip has a thermochromic substance impregnated into the substrate or dispersed within the adhesive composition. An increase in temperature causes a color change in the thermochromic substance. U.S. Pat. No. 5,340,537 issued to Big Three Industries on Aug. 23, 1994 discloses a temperature indicating composition which provide a color change to coatings containing the composition upon exposure to a predetermined heat history. U.S. Pat. No. 5,501,945 issued to the University of Akron, on Mar. 26, 1996 discloses a method of incorporating multi-chromic polymers into packaging which respond to exposure to specific stimuli, such as temperature and radiation, by a reversible change or shift in the frequencies of light which they adsorb. U.S. Pat. No. 4,421,560 issued to Pilot Ink Company Ltd. Dec. 20, 1983 discloses a thermochromatic material which undergoes reversible metachromatism at a temperature within the range of −50° C. to 120° C. The teachings of each of the foregoing patents are incorporated herein by reference to the extent that each describes an indicator suitable for use with the present invention. In an alternate embodiment, the indicator of the present invention may be an audible pressure relief valve.

While particular elements, embodiments and applications of the present invention have been described herein, it should be understood that the invention is not limited thereto since modifications may be made by those skilled in the art. It is therefore contemplated by the appended claims to cover such modifications that incorporate those features coming within the scope of the invention.

What is claimed is:

1. A microwaveable container for sterilizing objects comprising a sealable container for holding said objects, and an indicator integral with or attached to said sealable container; wherein said indicator is designed to indicate sufficient sterilization of said objects.

2. The container of claim 1, wherein said container comprises a housing having top and bottom portions which define an interior chamber, wherein said interior chamber is capable of communicating with the external environment when said top or bottom portion is placed in an open position; said internal chamber is sufficiently incapable of communicating with the external environment when said top or bottom portion is placed in a closed position.

3. The container of claim 1, further comprising an o-ring, seal or equivalent.

4. The container of claim 1, wherein said object is a medical device.

5. The container of claim 4, wherein said device is designed to be inserted into a body cavity or contact bodily fluids.

6. The container of claim 1, wherein said object is a household device.

7. The container of claim 6, wherein said household device is porous.

8. The container of claim 7, wherein said household device is absorbent.

9. The container of claim 1, wherein said object is a dental device

10. The container of claim 1, wherein said container is constructed of a material at least partially transparent to microwave energy.

11. The container of claim 10, wherein said material is further defined as being substantially impermeable to moisture

12. The container of claim 10, wherein said material is a polymer.

13. The container of claim 1, wherein said indicator is thermochromic.

14. The container of claim 13, wherein said indicator is attached to said container.

15. The container of claim 13, wherein said indicator is incorporated into said container.

16. The container of claim 1, wherein said indicator capable of emitting an audible sound at or above a predetermined temperature or pressure.

17. The container of claim 1, further comprising a member having a plurality of recesses and upstanding sides, for receiving or supporting one or more said objects placed within said interior chamber, said member being removable from said interior chamber.

18. The container of claim 1, further comprising an internal lining of microwave-absorbing material.

19. A method of sterilizing an object in a microwave oven comprising:
a). placing a contaminated material into a container made of a material at least partially transparent to microwave energy;
b). sealing said container; and
c). irradiating said container containing said contaminated material with said microwave energy.

20. The method of claim 19, further comprising placing said container and the material contained therein into a microwave oven and creating a microwave energy field inside said oven;

21. The method of claim 19, further comprising removing said container from said microwave when an indicator device is activated.

22. The method of claim 19, wherein said object is a medical device.

23. The container of claim 19, wherein said device is designed to be inserted into a body cavity or contact bodily fluids.

24. The method of claim 19, wherein said object is a household device.

25. The method of claim 24, wherein said household device is porous.

26 The method of claim 24, wherein said household device is absorbent.

27. The method of claim 19, wherein said object is a dental device

28. The method of claim 19, wherein said sterilization effectively kills infectious microorganisms inhabiting said object.

29. The method of claim 19, wherein said indicator is thermochromic.

30. The method of claim 19, wherein said indicator is capable of emitting an audible sound at or above a predetermined temperature or pressure.

31. A microwaveable container for sterilizing objects comprising:
a). a housing having top and bottom portions which define an interior chamber, wherein said interior chamber is capable of communicating with the external environment when said top or bottom portion is placed in an open position; said internal chamber is sufficiently incapable of communicating with the external environment when said top or bottom portion is placed in a closed position;
b). a receptacle for holding an amount of sterilizing fluid; and
c). an indicator for determining when an object placed within said container has been adequately sterilized.

32. The container of claim 31, further comprising a vent for releasing steam.

33. The container of claim 32, wherein said releasing of steam generates an audible acoustic signal.

34. The container of claim 33, wherein said signal indicates that objects placed within said container have been sterilized.

35. The container of claim 31, wherein said object is a medical device.

36. The container of claim 31, wherein said device is designed to be inserted into a body cavity or contact bodily fluids.

37. The container of claim 31, wherein said object is a household device.

38. The container of claim 37, wherein said household device is porous.

39. The container of claim 37, wherein said household device is absorbent.

40. The container of claim 31, wherein said object is a dental device

41. The container of claim 31, wherein said sterilizing fluid is water.

42. The container of claim 31, wherein said indicator is activated by change in pressure.

43. The container of claim 31, wherein the indicator is activated by a change in temperature.

44. The container of claim 31, further comprising a member having a plurality of recesses and upstanding sides, for receiving or supporting one or more said objects placed within said interior chamber, said member being removable from said interior chamber.

45. A method for sterilizing medical devices and household goods in a microwave oven comprising placing one or more contaminated devices in a microwaveable container, exposing said container to microwave radiation for a time period sufficient to generate a temperature inside said container capable of sterilizing said object, wherein said microwaveable container comprises an indicator placed on or incorporated into said container, said indicator being enabled to produce a visual or auditory signal when an object has been adequately sterilized.
